(19) United States
(12) Patent Application Publication
(10) Pub. No.: US 2005/0113573 A1
(43) Pub. Date: May 26, 2005

(54) Nitroso compounds to treat ischemia

(76) Inventors: Luis H. Toledo, Portage, MI (US); Edward J. Hessler, Kalamazoo, MI (US); Harold A. Karnes, Portage, MI (US)

Correspondence Address: VAN DYKE, GARDNER, LINN AND BURKHART, LLP, 2851 CHARLEVOIX DRIVE, S.E., P.O. BOX 888695, GRAND RAPIDS, MI, 49588-8695, US

(21) Appl. No.: 10/719,621

(22) Filed: Nov. 21, 2003

Related U.S. Application Data
(60) Provisional application No. 60/430,545.

(51) Int. Cl. C07D 413/14; C07D 43/14

(52) U.S. Cl. 540/601; 544/295; 544/114

(57) ABSTRACT

The invention includes piperazinyl pyrimidinyl nitroso compounds of the formula (II) 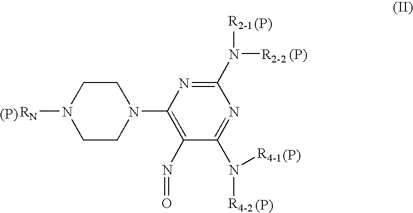 and N-Nitrosometoprolol and pharmaceutically acceptable salts thereof which are useful in treating one or more of the following ischemic diseases, coronary heart disease, stroke, hemorrhagic shock, peripheral vascular disease (of both the upper and lower extremities), abdominal vascular insufficiency and transplant related surgery.

### CROSS-REFERENCE TO RELATED APPLICATIONS

This patent application claims the benefit of U.S. provisional patent application Ser. No. 60/430,545, filed Dec. 3, 2002 under 35 U.S.C. §119(e)(i).

### BACKGROUND OF THE INVENTION

1. Field of the Invention

The present invention is nitroso compounds useful in treating ischemic diseases.

2. Description of the Related Art

U.S. Pat. Nos. 5,099,019 and 5,175,281 discloses 2,4-di(1-pyrrolidinyl)-6-(1-piperazinyl)pyrimidine, see Preparation A-22. In addition, these patents disclose other 2,4-di(substituted)aminopiperazinylpyrimidines.

The Physicians Desk Reference (PDR), discloses metoprolol is a β-adrenergic agent useful in treating hypertension, angina pectoris and myocardial infarction.

The Physicians Desk Reference (PDR), discloses carvediol is a β-adrenergic agent useful in treating hypertension and heart failure.

Ischemic diseases are diseases caused by a lack of blood supply and include coronary heart disease, stroke, hemorrhagic shock, peripheral vascular disease (of both the upper and lower extremities), abdominal vascular insufficiency and transplant related surgery. These diseases are the most frequent cause of morbidity and mortality in the industrialized countries. Interruption of blood supply results in ischemia, which activates leukocytes particularly neutrophils, as well as platelets, and rapidly damages metabolically active tissues. Without cooling of the organ, irreversible damage will occur within a matter of minutes depending on the organ type. Paradoxically, restoration of blood flow initiates a cascade of events that may lead to additional cell injury.

At present there are few pharmaceutical agents to treat these ischemic diseases. The agents available include nitrites, nitrates, various non-steroidal anti-inflammatory derivative (NSAIDs) analgesics and agents to treat platelet effects. However, none of these agents are completely effective, they do not fully prevent (transplant) or reduce (after an ischemic event) the damage caused by the ischemic event. The compounds of the invention effectively treat these diseases.

The Journal of investigative Surgery, 14, 267-273 (2001) discloses that both exogenous and endogenous nitric oxide (NO) improves the function and survival of ischemically injured livers. It is known that L-arginine catabolism to L-citrulline is catalyzed by a family of nitric oxide synthases and produces nitric oxide. Therefore, L-arginine was tested along with sodium nitroprusside. The authors measured liver function, neutrophil infiltration and animal survival after liver ischemia/reperfusion injury. The authors found that both exogenous donors (sodium nitroprusside) and substrates for the endogenous pathway (L-arginine) were beneficial for the liver after severe ischemia/reperfusion.

Transplantation, 70(10) 1431-1437 (2000) reported that nitric oxide, given as sodium nitroprusside, demonstrated a protective effect in treating ischemic injury of the kidney in rats which had been subjected to 75 min of renal warm ischemia and contralateral nephrectomy.

Journal of Surgical Research, 105, 248-258 (2002) is a research review which discloses that nitric oxide is a free radical and a mediator of ischemia and reperfusion injury. Further, that nitric oxide is synthesized from the guanidino group of L-arginine by nitric oxide synthases and the nitric oxide synthesized produces organ damage. The authors also report that nitric oxide can act in a tissue-protective manner by scavenging oxygen-derived free radicals and other means. The authors report that there are three strategies to prevent ischemia and reperfusion injury which are, nitric oxide supplementation, antioxidant molecules and neutrophil-endothelial cell blockade. With regard to nitric oxide supplementation, a number of different approaches are reviewed. These include nitric oxide gas, nitric oxide dissolved in normal or physiological saline, nitric oxide in low concentrations, nitric oxide donors and administration of L-arginine. With regard to nitric oxide donors most were organic nitrates.

Journal of Surgical Research, 87, 201 (1999) reports that with regard to overcoming ischemia and/or reperfusion injury in organ transplantation a number of compounds have been tried. More particularly, sodium nitroprusside was added to University of Wisconsin (UW) solution during liver cold storage resulting in improvement in intrahepatic circulation, an increase in bile production and better histological appearance of the organ.

Free Radicial Biology & Medicine, 28(10), 1495-1506 (2000) discussed nitric oxide related pharmaceuticals and their approach to therapeutic nitric oxide modulation. Most nitric oxide insufficiency diseases so far identified are cardiovascular, in particular hypertension. In an attempt to treat cardiovascular diseases, such as restenosis, a number of nitric oxide donors representing many different chemical classes have been tried in vivo. These include, organic nitrates such as glyceryl trinitrate, organic nitrites, diazenium diolates (NONOates) such as spermine NONOate, sydnonimines such as linsidomine and molsidomine, and nitrosothiols such as S-Nitrosoglutathione. However, none of these agents are approved by the FDA for treating ischemic diseases.

Transplantation, 66(8), 994-9 (1998) discloses that the nitric oxide donor sodium nitroprusside was protective in ischemia/reperfusion injury of the pancreas in pigs. The problems associated with sodium nitroprusside were discussed above.

J. Thorac. Cardiovasc. Surg., 111(4), 882-92 (1996) reports on the results of a study of sodium nitroprusside in 16 children undergoing cardiopulmonary bypass surgery. The results indicated that sodium nitroprusside had an inhibiting effect on complement activation by nitric oxide release.

Circulatory Shock, 44, 91-95 (1995) reported on the role of nitric oxide in ischemia/reperfusion of the rat kidney and concluded that sodium nitroprusside gave significantly better survival with minimal histological damage. The authors' conclusion was that exogenous nitric oxide has a beneficial and protective effect of the ischemically damaged rat kidney.

Transplantation, 61(2) 179-183 (1996) reported on the time dependence of sodium nitroprusside administration in the prevention of neutrophil infiltration in the rat ischemic kidney. The authors concluded that IV sodium nitroprusside gave significant improvement in all parameters measured when administered at 75, 30 and 15 min prior to reperfusion. When administered at 5 min prior to reperfusion, the improvement was not observed.

Hepato-Gastroenterology, 47, 1722-1725 (2000) reported on possible therapeutic strategies regarding nitric oxide and hepatic ischemic-reperfusion injury. The authors concluded that in animal models, therapeutic strategies that increase endogenous nitric oxide concentrations in the liver significantly decrease reperfusion injury. They concluded, that such treatment modalities may have important clinical implications for the future, particularly in view of the increasing use in hepatic transplantation programs of marginal donor livers with their greater susceptibility to ischemia-reperfusion injury. Exogenous sources of nitric oxide were not discussed.

American Journal of Physiology, 276, GI1313-G1316 (1999) after reviewing the work of others, the authors concluded that compounds which release nitric oxide in small amounts over a prolonged period of time may be useful for prevention of gastrointestinal injury associated with shock and with the use of pharmaceuticals that have ulcerogenic effects. More specifically, Gastroenterology, 107, 173-179 (1994) experimented with compounds which had a nitric oxide-releasing moiety attached to standard nonsteroidal anti-inflammatory drugs (NSAIDs), flurbiprofen and ketoprofen. The results indicated the compounds had markedly reduced ulcerogenic properties. Neither the compounds of the invention nor their parents are NSAIDs.

The Journal of Neurology, Neurosurgery & Psychiatry, 67, 1-3 (1999) in an editorial discussed the possible role of nitric oxide in acute ischemic stroke. Based on the work reported in Trends Neurosci., 20, 132-9 (1997) the editorial reported that enhanced nitric oxide release with eNOS, L-arginine and sodium nitroprusside results in smaller cerebral infarcts than those in vehicle treated animals.

Therefore, numerous agents have been proposed, and made, which release nitrous oxide to treat ischemic diseases. The only two types of agents on the market for treating ischemic diseases which release nitric oxide are nitrates such as nitroglycerin and isosorbide, and sodium nitroprusside. However, the nitrates can produce severe hypotension, syncope, arrhythmias, headaches, dizziness and other severe side effects. Sodium nitroprusside contains a cyano group and therefore is toxic. Another disadvantage of sodium nitroprusside is that it has a toxic hypotensive effect. At present there are no agents that completely reverse the ischemic disease and are non-toxic. The compounds of the invention have neither a cyano group nor a toxic hypotensive effect as seen with the other compounds.

Circulation, 87(2), 590-7 (1993) studied the effects of a nitric oxide donor, 3-morpholino-sydnonimine on platelet adhesion and platelet-thrombus formation in pigs. The authors concluded that the antiadhesive effects of 3-morpholino-sydnonimine indicated that a nitric oxide donor may prove effective in modifying the pathophysiological response to angioplasty injury.

Circulation, 95(1), 83-9 (1997) reported on a study of 700 stable coronary patients scheduled for angioplasty. The patients were treated with the nitric oxide donors linsidomine and molsidomine before the angioplasty. The results indicated a modest improvement in the long-term angiographic result but there was no effect on the clinical outcome.

Cardiovascular Res., 30(1), 87-96 (1995) reported on a study of a nitric oxide donor, molsidomine, in pigs with regard to smooth muscle proliferation following carotid angioplasty. The authors found that exogenous nitric oxide inhibits smooth muscle cell proliferation following balloon angioplasty in pigs. The authors concluded that the inhibitory effects of nitric oxide on platelet adhesion and smooth muscle proliferation identified a possible role for high local concentrations of nitric oxide to modify the vascular response to balloon angioplasty.

Radiology, 219, 716-723 (2001) discloses a comparative trial using L-arginine, r-hirudin and molsidomine with regard to the compounds ability to reduce restenosis after balloon angioplasty in rabbit iliac arteries. The results indicated that L-arginine and molsidomine, but not r-hirudin, caused significant reduction of the neointimal area.

Heart, 86, 368-372 (2001), a review, was concerned with the role of nitric oxide in cardioprotection. They discussed a number of nitric oxide donors including linsidomine, molsidomine, isosorbide diniitrate, L-arginine, SPM-5185 (a cysteine containing nitric oxide donor), glyceryl trinitrate, SIN-1 (hepatic metabolite of molsidomine, available in systemic form as linsidomine), SNP a nitroprusside dianion consisting of a complex of ferrous ion with five cyanide ions, SNAP a nitrosothiol that directly releases nitric oxide slowly without prior biotransformation, DELTA-NO a diazenium diolate with a 20 hour half-life, and NO aspirins which is a new class nitric oxide donors attached to the aspirin molecule. Yet one of the authors' conclusions was that, “What is first needed is the development of more refined pharmacological NO donors.”

J. Clin. Invest., 96(6), 2630-8 (1995) reports on a study in rabbits using a protein adduct of nitric oxide with regard to inhibition of neointimal proliferation. The nitric oxide adduct was S-nitrososerum albumin a naturally occurring adduct of nitric oxide which has a prolonged biological half-life. The study investigated the effects of locally delivered S-nitroso-bovine serum albumin and a poly thiolated form of bovine serum albumin modified to include several S-nitrosothiol groups. The authors found that local administration inhibited intimal proliferation and platelet deposition after vascular arterial balloon injury. They concluded that the strategy for local delivery of a long-lived nitric oxide adduct has potential for preventing restenosis after angioplasty.

Lancet, 338(8776) 11734 (1991), reported on inhaled nitric oxide and pulmonary vasodilatation. The compounds of the present invention are not inhaled and are not used for pulmonary vasodilatation.

Arteriosclerosis, Thrombosis and Vascular Biology, 22, 263 (2002) studied a structural derivative of flurbiprofen containing an added nitric oxide releasing moiety termed HCT-1026 with effects in a rat with regard to vascular injury and restenosis. The authors found a reduction in the neointimal proliferation which correlated with an increase in nitrite/nitrate plasma levels and reduced cell proliferation. They concluded that the reduction in restenosis appeared to be directly related to nitric oxide release.

Laboratory Investigation, 82, 825-832 (2002) reported on the study of prolonged treatment of nitric oxide-releasing form of aspirin and the influence on femoral arteries of hypercholesterolemic mice. They found that nitric oxide releasing aspirin reduced the vascular inflammation and promoted apoptosis during vascular remodeling associated with neointimal thickening. The compounds of the present invention are not at all related to aspirin.

British Journal of Pharmacology, 137, 295-310 (2002) reviewed the pharmacology and potential therapeutic applications of nitric oxide-releasing non-steroidal anti-inflammatory and related nitric oxide-donating pharmaceuticals including nitroflurbiprofen, nitronaproxen, nitropednisolone, nitrofenac, nitrodiclofenac, nitroifuprofen, nitroindomethacin and others. They found that the slow release of nitric oxide from these compounds led to subtle changes in the profile of the pharmacological activity of the parent non-steroidal anti-inflammatory agent. The parent compound of the nitric oxide of the present invention is known only as an intermediate in producing 21-aminosteroids.

NitroMed is a company which is trying to prepare nitric oxide pharmaceuticals which when taken in vivo release nitric oxide (NO). They have in development NO-aspirin, NO-diclofenac, NO-naproxen, NO-ketoprofen, NO-ibuprofen and S-NO-diclofenac. None of those compounds is similar to the piperazinyl pyrimidinyl nitroso compounds (II) of the present invention.

Nicox, a French company, is also involved in developing nitric oxide releasing compounds by attaching nitric oxide onto existing pharmaceuticals. These compounds include nitric oxide derivatives of aspirin, flurbiprofen, hydrocortisone, prednisolone, salbutamol, budesonide, ursodeoxycholic acid, sildenafil and sulindac. None of these compounds are similar to the compounds of this invention.

U.S. Pat. No. 6,255,277 discloses a method of preventing an adverse effect associated with the use of a medical device in a patient comprising introducing into the patient a medical device of which at least a portion comprises a nitric oxide adduct, wherein the nitric oxide adduct comprises an S-Nitroso-protein.

### SUMMARY OF INVENTION

Disclosed are piperazinyl pyrimidinyl nitroso compounds of the formula (II) 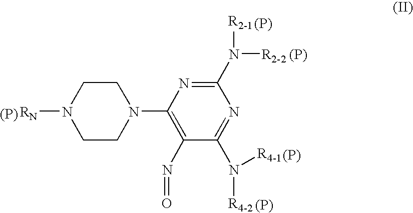
where (P)—R_{N} is:
   —N═O,
   (P)—R_{N-1}—OC—(CH₂)ₙ₁— where n₁ is 1 thru 6 and where (P)—R_{N-1} is H— or C₁-C₄ allcyl,
   C₁-C₆ alkyl,
where (P)—R₂₋₁ is:
   —N═O and
   C₁-C₆ alkyl;
where (P)—R₂₋₂ is:
   C₁-C₆ alkyl; and
where (P)—R₂₋₁ and (P)—R₂₋₂ are taken together with the attached nitrogen atom to form a ring selected from the group consisting of:
   pyrrolidinyl,
   piperidinyl,
   homopiperidinyl,
   morpholinyl,
   4-nitroso-1-piperazinyl;
where (P)—R₄₋₁ is
   —N═O and
   C₁-C₆ alkyl; and
where (P)—R₄₋₂ is
   C₁-C₆ alkyl; and
where (P)—R₄₋₁ and (P)—R₄₋₂ are taken together with the attached nitrogen atom to form a ring selected from the group consisting of:
   pyrrolidinyl,
   piperidinyl,
   homopiperidinyl,
   morpholinyl,
   4-nitroso-1-piperazinyl; and pharmaceutically acceptable salts thereof.

Also disclosed is a N-nitrosometoprolol compound of formula (IV) 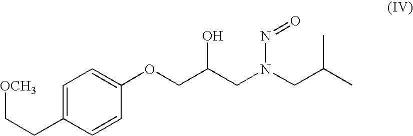
and pharmaceutically acceptable salts thereof.

Further disclosed is a method of treating a human who has an ischemic disease selected from the group consisting of coronary heart disease, stroke, hemorrhagic shock, peripheral vascular disease (upper and lower extremities) and transplant related injuries and who is in need of treatment which comprises administering to that human an anti-ischemic effective amount of a piperazinyl pyrimidinyl nitroso compound of formula (II) 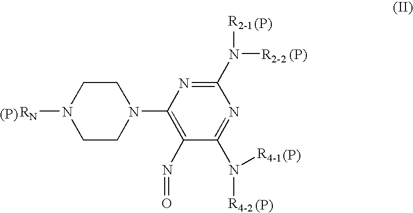
where (P)—R_{N}, (P)—R₂₋₁, (P)—R₂₋₂, (P)—R₄₋₁ and (P)-R₄₋₂ are as defined above and N-nitrosometoprolol of formula (IV) 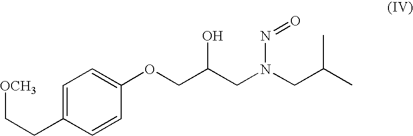
   and pharmaceutically acceptable salts thereof.

### DETAILED DESCRIPTION OF THE INVENTION

“Nitroso Compounds” refers to the piperazinyl pyrimidinyl nitroso compounds (II), N-nitrosometoprolol (IV). The “nitroso compounds” of the invention are prepared from known starting materials, or from compounds which can be readily be prepared from known compounds by those skilled in the art, by means known to those skilled in the art.

In the compounds below the term “(SM)-Rₓ” or “—Rₓ (SM)” refers to the variable substituent Rₓ in the starting material (SM-) and “(P)—Rₓ” or “—Pₓ(P) refers to the same variable substituent but in the product (P—). For some variable substituents there is no change, for others such as a hydrogen atom on a secondary amine, they can become nitroso groups, —N═O.

The piperazinyl pyrimidinyl nitroso compounds (II) 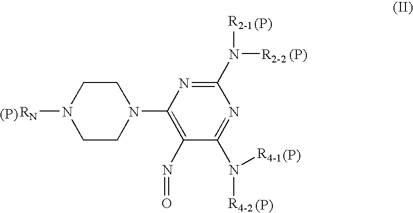
where (P)—R_{N} is:
   —N═O,
   (P)—R_{N-1}—O—OC—(CH₂)ₙ₁— where n₁ is 1 thru 6 and where (P)—R_{N-1} is H— or C₁-C₄ alkyl,
   C₁-C₆ alkyl,
where (P)—R₂₋₁ is:
   —N═O and
   C₁-C₆ alkyl;
where (P)—R₂₋₂ is:
   C₁-C₆ alkyl; and
where (P)—R₂₋₁ and (P)—R₂₋₂ are taken together with the attached nitrogen atom to form a ring selected from the group consisting of:
   pyrrolidinyl,
   piperidinyl,
   homopiperidinyl,
   morpholinyl,
   4-nitroso-1-piperazinyl;
where (P)—R₄₋₁ is
   —N═O and
   C₁-C₆ alkyl; and
where (P)—R₄₋₂ is
   C₁-C₆ alkyl; and
where (P)—R₄₋₁ and (P)—R₄₋₂ are taken together with the attached nitrogen atom to form a ring selected from the group consisting of:
   pyrrolidinyl,
   piperidinyl,
   homopiperidinyl,
   morpholinyl,
   4-nitroso-1-piperazinyl are prepared from the corresponding piperazinyl pyrimidinyl compounds of formula (I)
where (SM)-R_{N} is:
   —H,
   (SM)-R_{N-1}—O—OC—(CH₂)ₙ₁— where n₁ is 1 thru 6 and where (SM)-R_{N-1} is H— or C₁-C₄ alkyl,
   C₁-C₆ alkyl,
where (SM)-R₂₋₁ is:
   —H and
   C₁-C₆ alkyl;
where (SM)-R₂₋₂ is:
   C₁-C₆ alkyl; and
where (SM)-R₂₋₁ and (SM)-R₂₋₂ are taken together with the attached nitrogen atom to form a ring selected from the group consisting of:
   pyrrolidinyl,
   piperidinyl,
   homopiperidinyl,
   morpholinyl,
   1-piperazinyl;
where (SM)-R₄₋₁ is
   —H and
   C₁-C₆ alkyl; and
where (SM)-R₄₋₂ is
   C₁-C₆ alkyl; and
where (SM)-R₄₋₁ and (SM)-R₄₋₂ are taken together with the attached nitrogen atom to form a ring selected from the group consisting of:
   pyrrolidinyl,
   piperidinyl,
   homopiperidinyl,
   morpholinyl,
   1-piperazinyl.

The process is the standard method of producing N-nitroso compounds. The starting material, the piperazinyl pyrimidinyl compounds (I), are mixed with aqueous acid. The nature of the aqueous acid is not important, hydrochloric, sulfuric and phosphoric are all operable. Nitrite, NO₂⁻, in the form of an aqueous solution, preferably either sodium or potassium nitrite, is then added drop wise with stirring to the piperazinyl pyrimidinyl (I)-acid mixture. The mixture is then stirred for some additional time, from about 10 min to about 2 hr, usually about 0.5 to about 1 hr. The reaction can be performed at room temperature (20-25°), however, it is preferred to add the nitrite solution at about 40° and then stir at 20-25°. When the reaction is complete as determined by TLC, or other convenient means, the desired piperazinyl pyrimidinyl nitroso product (II) is obtained by extraction with an organic water immiscible solvent, such as ethyl acetate, as is know to those skilled in the art. While performing the extraction it is preferred to add a weak base such as bicarbonate or carbonate. A 5% solution of potassium bicarbonate works well.

Similarly, starting with metoprolol (III) gives the corresponding N-nitrosometoprolol (IV).

By the process of this invention, secondary amines, RₐR_{b}N—H, are transformed to the corresponding N-nitroso compounds and therefore RₐR_{b}N—H will be transformed to the corresponding nitroso compound RₐR_{b}N—N═O. If Rₐ and R_{b} are cyclized to form a ring, the N will still form a nitroso compound. Therefore, when the piperazinyl pyrimidinyl compound (I) or carvedilol (III) starting material contains more than one secondary amine, then the “nitroso compounds” produced can contain more than one nitroso group. In addition, in the piperazinyl pyrimidinyl compounds (I), the C-5-CH═ group of the pyrimidinyl moiety is nitrosated.

Given the fact that the piperazinyl pyrimidinyl compounds (I) can be nitrosated at the pyrimidinyl C-5 carbon and at a number of secondary amine positions, there are may different possibilities for having one or more nitroso groups added to the piperazinyl pyrimidinyl compounds (I). For example, when (SM)-R₂₋₁ is —H, then the group —N[(SM)-R₂₋₁][(SM)-R₂₋₂] is —N(—H)(C₁-C₆ alkyl) the product will be —N(—N═O)(C₁-C₆ alkyl) giving an additional nitroso group. Likewise, when —N[(SM)-R₂₋₁][(SM)-R₂₋₂] is cyclized to form piperazinyl, the piperazinyl group will be nitrosated to form -piperazinyl-N═O. In addition, when (SM)-R_{N}— is H—, that position of the piperazinyl is a secondary amine and will be nitrosated. If both —N[(SM)-R₂₋₁][(SM)-R₂₋₂] and —N[(SM)-R₄₋₁][(SM)-R₄₋₂] are secondary amines, then positions will be converted to —N═O in addition to the C-5 pyrimidinyl position. Therefore, the piperazinyl pyrimidinyl nitroso compound (II) can have one, two, three or four nitroso groups depending on the substitution of the piperazinyl pyrimidinyl starting compound (I). If one desires a piperazinyl pyrimidinyl nitroso compound (II) with just one or two nitroso groups it is preferred that (SM)-R₂₋₁ and/or (SM)-R₄₋₁ are not —H nor cyclized to form piperazinyl. One way to control the number of nitroso groups piperazinyl pyrimidinyl nitroso compound (II) is to control the number of secondary amine groups in the piperazinyl pyrimidinyl starting compound (I). It is easier to control the number of nitroso groups in the piperazinyl pyrimidinyl nitroso compound (II) by controlling the number of secondary amines in the piperazinyl pyrimidinyl starting compound (I) than by controlling the number of equivalents of sodium nitrite added.

The piperazinyl pyrimidinyl nitroso compounds (II) and N-nitrosometoprolol (IV) are amines and as such form pharmaceutically acceptable salts with pharmaceutically acceptable acids. Pharmaceutically acceptable salts include salts of both inorganic and organic acids. The pharmaceutically acceptable salts are preferred over the corresponding free amines since they produce compounds which are more water soluble and more crystalline. Pharmaceutically acceptable salts are preferred over the corresponding free bases since they produce compounds which are more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include salts of both inorganic and organic acids. The preferred pharmaceutically acceptable salts include salts of the following acids acetic, aspartic, benzenesulfonic, benzoic, bicarbonic, bisulftric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfiric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfiric, tannic, tartaric, teoclic and toluenesulfonic. For other acceptable salts, see Int. J. Pharm., 33, 201-217 (1986) and J. Pharm. Sci., 66(1), 1, (1977)

In treating the ischemic diseases coronary heart disease, stroke, hemorrhagic shock, peripheral vascular disease (upper and lower extremities) and transplant related injuries, for acute treatment, the piperazinyl pyrimidinyl nitroso compounds (II) and N-nitrosometoprolol (IV) and pharmaceutically acceptable salts thereof, are given initially IV over a period of about 24 hr, preferably a dose every 8 hr for three doses. If needed, the administration can be continued IV. However, if the patient is able to tolerate oral administration, then the piperazinyl pyrimidinyl nitroso compounds (II) and N-nitrosometoprolol (IV) can be administered orally.

When given IV, the piperazinyl pyrimidinyl nitroso compounds (II) and N-nitrosometoprolol (IV) and pharmaceutically acceptable salts are compounded into a sterile aqueous solution, with pH adjusted appropriately as is known to those skilled in the art. When given orally, the usual oral dosage forms of tablets, capsules, suspension, solution, emulsion are all useful. The piperazinyl pyrimidinyl nitroso compounds (II) and N-nitrosometoprolol (IV) and pharmaceutically acceptable salts thereof are compounded into known parenteral and oral dosage forms in the usual manner as is known to those skilled in the art. The invention here is not the pharmaceutical dosage forms but rather the compounds themselves and the manner in which they are used. When given IV, the dose is from about 5 to about 100 mg/kg/dose (one to four doses daily), preferably from about 25 to about 50 mg/kg/dose. When given orally, the dose is from about 5 to about 50 mg /kg/dose; preferably from about 10 to about 20 mg/kg/dose. When given orally, one to four doses are given daily.

When the ischemic disease is transplant related injuries, then the piperazinyl pyrimidinyl nitroso compounds (II) and N-nitrosometoprolol (IV) and pharmaceutically acceptable salts thereof can be given preventively before the surgery. In that situation, the piperazinyl pyrimidinyl nitroso compounds (II) and N-nitrosometoprolol (IV) and pharmaceutically acceptable salts thereof are given starting about 48 hr before surgery and during the surgery. The dose is the same as for treating an acute ischemic disease.

The exact dosage and frequency of administration depends on the particular piperazinyl pyrimidinyl nitroso compounds (II) and N-nitrosometoprolol (IV) used, the particular condition being treated, the severity of the condition being treated, the route of administration, the age, weight, general physical condition of the particular patient, other medication the individual may be taking as is well known to those skilled in the art. In addition, the exact dosage and frequency of administration can be more accurately determined by measuring the blood level or concentration of the piperazinyl pyrimidinyl nitroso compounds (II) and N-nitrosometoprolol (IV) in the patient's blood and/or the patients' response to the particular condition being treated.

### DEFINITIONS AND CONVENTIONS

The definitions and explanations below are for the terms as used throughout this entire document including both the specification and the claims.

### I. Conventions for Formulas and Definitions of Variables

The chemical formulas representing various compounds or molecular fragments in the specification and claims may contain variable substituents in addition to expressly defined structural features. These variable substituents are identified by a letter or a letter followed by a numerical subscript, for example, “Z₁” or “Rᵢ” where “i” is an integer. These variable substituents are either monovalent or bivalent, that is, they represent a group attached to the formula by one or two chemical bonds. For example, a group Z₁ would represent a bivalent variable if attached to the formula CH₃—C(=Z₁)H. Groups Rᵢ and Rⱼ would represent monovalent variable substituents if attached to the formula CH₃—CH₂—C(Rᵢ)(Rⱼ)H₂. When chemical formulas are drawn in a linear fashion, such as those above, variable substituents contained in parentheses are bonded to the atom immediately to the left of the variable substituent enclosed in parenthesis. When two or more consecutive variable substituents are enclosed in parentheses, each of the consecutive variable substituents is bonded to the immediately preceding atom to the left which is not enclosed in parentheses. Thus, in the formula above, both Rᵢ and Rⱼ are bonded to the preceding carbon atom. Also, for any molecule with an established system of carbon atom numbering, such as steroids, these carbon atoms are designated as Cᵢ, where “i” is the integer corresponding to the carbon atom number. For example, C₆ represents the 6 position or carbon atom number in the steroid nucleus as traditionally designated by those skilled in the art of steroid chemistry. Likewise the term “R₆” represents a variable substituent (either monovalent or bivalent) at the C₆ position.

Chemical formulas or portions thereof drawn in a linear fashion represent atoms in a linear chain. The symbol “—” in general represents a bond between two atoms in the chain. Thus CH₃—O—CH₂—CH(Rᵢ)—CH₃ represents a 2-substituted-1-methoxypropane compound. In a similar fashion, the symbol “═” represents a double bond, e.g., CH₂═C(Rᵢ)—O—CH₃, and the symbol “≡—” represents a triple bond, e.g., HC≡C—CH(Rᵢ)—CH₂—CH₃. Carbonyl groups are represented as —CO—.

The carbon atom content of variable substituents is indicated in one of two ways. The first method uses a prefix to the entire name of the variable such as “C₁-C₄”, where both “1” and “4” are integers representing the minimum and maximum number of carbon atoms in the variable. The prefix is separated from the variable by a space. For example, “C₁-C₄ alkyl” represents alkyl of 1 through 4 carbon atoms, (including isomeric forms thereof unless an express indication to the contrary is given). Whenever this single prefix is given, the prefix indicates the entire carbon atom content of the variable being defined. Thus C₂-C₄ alkoxy-carbonyl describes a group CH₃—(CH₂)ₙ-0-CO— where n is zero, one or two. By the second method the carbon atom content of only each portion of the definition is indicated separately by enclosing the “Cᵢ-Cⱼ” designation in parentheses and placing it immediately (no intervening space) before the portion of the definition being defined. By this optional convention (C₁-C₃)alkoxycarbonyl has the same meaning as C₂-C₄ alkoxycarbonyl because the “C₁-C₃” refers only to the carbon atom content of the alkoxy group. Similarly while both C₂-C₆ alkoxyalkyl and (C₁-C₃)alkoxy(C₁-C₃)alkyl define alkoxyalkyl groups containing from 2 to 6 carbon atoms, the two definitions differ since the former definition allows either the alkoxy or alkyl portion alone to contain 4 or 5 carbon atoms while the latter definition limits either of these groups to 3 carbon atoms.

### II. Definitions

All temperatures are in degrees Celsius.

Metoprolol refers to 1-[4-(2-methoxyethyl)phenoxy]-3-[(1-methylethyl)amino]-2-propanol.

“Nitroso Compounds” refers to the pyrimidinyl nitroso compounds (II) and N-nitrosometoprolol (IV).

IV refers to intravenous administration.

Chromatography (column and flash chromatography) refers to purification/separation of compounds expressed as (support, eluent). It is understood that the appropriate fractions are pooled and concentrated to give the desired compound(s).

NMR refers to nuclear (proton) magnetic resonance spectroscopy, chemical shifts are reported in ppm (δ) downfield from TMS.

MS refers to mass spectrometry expressed as m/e, m/z or mass/charge unit. [M+H]⁺ refers to the positive ion of a parent plus a hydrogen atom. EI refers to electron impact. CI refers to chemical ionization. FAB refers to fast atom bombardment.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### 5-Nitroso-2,4-di(1-pyrrolidinyl)-6-(4-nitroso-1-piperazinyl)pyrimidine (II)

2,4-Di(1-pyrrolidinyl)-6-(1-piperazinyl)pyrimidine (I, 111641-17-9, U.S. Pat. No. 5,099,019, Preparation A-22, 308 mg; 1 mmole) is added to a mixture of concentrated hydrochloric acid (1.0 ml) and water (4 ml) and is stirred at 40°. Sodium nitrite (831 mg; 12.03 mmole) is dissolved in water (4.0 ml). The nitrite solution is added drop wise over 15 min to the pyrimidine mixture. The reaction mixture is then stirred for about 30 min at 20-25°.

Ethyl acetate (50 ml) and potassium bicarbonate (50 ml of 5% solution) is added. The phases are separated and the aqueous phase is extracted with ethyl acetate (2×50 ml). The organic phases are combined, dried, filtered and concentrated to a foam. The foam is chromatographed (silica gel; methylene chloride/isopropyl alcohol, 95/5) to give the title compound, NMR (CDCl₃) 1.2 (impurity, isopropanol), 1.85, 2.0, 3.65, 3.85, 4.0 and 7.3 δ; MS=361.12(M+1).

### Example 2

### 2,4-Di(1-pyrrolidinyl)-6-[4-(3-propionic acid methyl ester) piperazin-1-yl]pyrimidine (intermediate)

2,4-Di(1-pyrrolidinyl)-6-(1-piperazinyl)pyrimidine (I, 0.7 g, 0.0023 moles), 3-bromopropionic acid methyl ester (0.7 g), sodium iodide (0.1 g), acetonitrile (20 ml) and triethylamine (5 ml) are mixed and stirred at 20-25° for 2 hr. The mixture is then lo concentrated, ethyl acetate 50 ml are added. The mixture is extracted saline (2×100 ml). The mixture is dried and concentrated under reduced pressure to dryness. The solid is crystallized from ether/hexane to give the title compound.

### Example 3

### 5-Nitroso-2,4-di(1-pyrrolidinyl)-6-[4-(3-propionic acid methyl ester)piperazin-1-yl]pyrimidine (II)

Following the general procedure of EXAMPLE 1 and making non-critical variations but starting with 2,4-di(1-pyrrolidinyl)-6-[4-(3-propionic acid methyl ester)piperazin-1-yl]pyrimidine (intermediate, EXAMPLE 2), the title compound is obtained, TLC (methylene chloride/methanol, 95/5)=0.4.

### Example 4

### N-Nitrosometoprolol (IV)

1-[4-(2-Methoxyethyl)phenoxy]-3-[(1-methylethyl)amino]-2-propanol (III, 3.0 mmoles) is added to water (6 ml) and isopropanol (3.5 ml) and hydrochloric acid (0.42 ml). The mixture is stirred at 40°. A solution of sodium nitrite (765 mg) in water (4 ml) is added drop wise and the mixture stirred for 1 hr at 20-25°. Potassium bicarbonate (20%, 25 ml) is added and the mixture is extracted with ethyl acetate (3×25 ml). The organic extracts are combined, dried, filtered and concentrated to give the title compound, NMR (CDCl₃) 1.3, 1.5, 2.1, 2.8, 3.4, 3.6, 3.8, 3.9, 4.1, 4.7, 6.9, 7.2 and 7.3 δ.

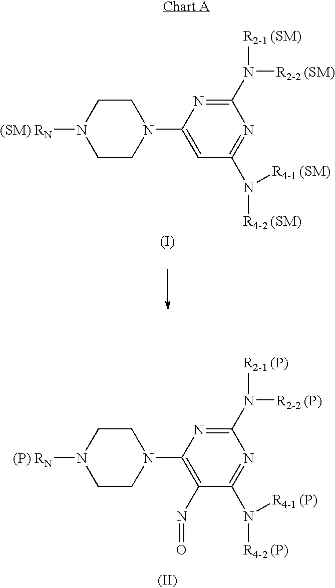

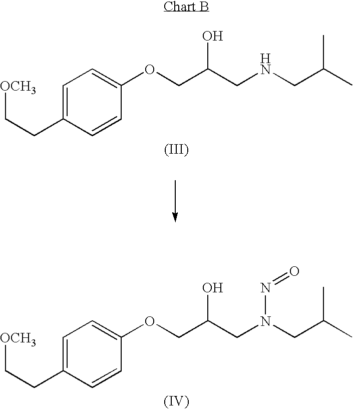

### ENUMERATED EMBODIMENTS

1. Use of a piperazinyl pyrimidinyl nitroso compound of the formula (II) 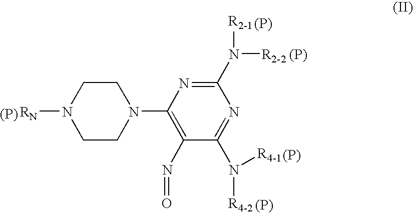
where P—R_{N} is:
   —N═O,
   P—R_{N-1}—O—OC—(CH₂)ₙ₁— where n₁ is 1 thru 6 and where P—R_{N-1} is H— or C₁-C₄ alkyl,
   C₁-C₆ alkyl,
where P—R₂₋₁ is:
   —N═O and
   C₁-C₆ alkyl;
where P—R₂₋₂ is:
   C₁-C₆ alkyl; and
where P—R₂₋₁ and P—R₂₋₂ are taken together with the attached nitrogen atom to form a ring selected from the group consisting of:
   pyrrolidinyl,
   piperidinyl,
   homopiperidinyl,
   morpholinyl,
   4-nitroso-1-piperazinyl;
where P-R₄₋₁ is
   —N═O and
   C₁-C₆ alkyl; and
where P—R₄₋₂ is
   C₁-C₆ alkyl; and
where P—R₄₋₁ and P—R₄₋₂ are taken together with the attached nitrogen atom to form a ring selected from the group consisting of:
   pyrrolidinyl,
   piperidinyl,
   homopiperidinyl,
   morpholinyl,
   4-nitroso-1-piperazinyl; or 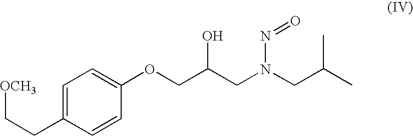
      and pharmaceutically acceptable salts thereof for the manufacture of a medicament for use in treating an ischemic disease selected from the group consisting of coronary heart disease, stroke, hemorrhagic shock, peripheral vascular disease (upper and lower extremities) and transplant related injuries.

1. A piperazinyl pyrimidinyl nitroso compound of the formula (II) 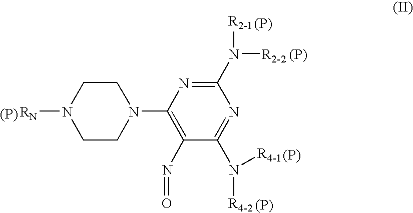
where (P)—R_{N} is:
—N═O,
(P)—R_{N-1}—O—OC—(CH₂)ₙ₁— where n₁ is 1 thru 6 and where (P)—R_{N-1} is H— or C₁-C₄ alkyl,
C₁-C₆ alkyl,
where (P)—R₂₋₁ is:
—N═O and
C₁-C₆ alkyl;
where (P)—R₂₋₂ is:
C₁-C₆ alkyl; and
where (P)—R₂₋₁ and (P)—R₂₋₂ are taken together with the attached nitrogen atom to form a ring selected from the group consisting of:
pyrrolidinyl,
piperidinyl,
homopiperidinyl,
morpholinyl,
4-nitroso-1-piperazinyl;
where (P)—R₄₋₁ is
—N═O and
C₁-C₆ alkyl; and
where (P)—R₄₋₂ is
C₁-C₆ alkyl; and
where (P)—R₄₋₁ and (P)—R₄₋₂ are taken together with the attached nitrogen atom to form a ring selected from the group consisting of:
pyrrolidinyl,
piperidinyl,
homopiperidinyl,
morpholinyl,
4-nitroso-1-piperazinyl; and pharmaceutically acceptable salts thereof.

2. A piperazinyl pyrimidinyl nitroso compound according to claim 1 where the variable substituents (P)—R₂₋₁ and (P)—R₂₋₂ are the same as the variable substituents (P)—R₄₋₁ and (P)—R₄₋₂.

3. A piperazinyl pyrimidinyl nitroso compound according to claim 1 where (P)—R₂₋₁ and (P)—R₂₋₂, and (P)—R₄₋₁ and (P)—R₄₋₂ are both taken together with the attached nitrogen atom to form pyrrolidinyl.

4. A piperazinyl pyrimidinyl nitroso compound according to claim 1 which contains 3 —N═O groups.

5. A piperazinyl pyrimidinyl nitroso compound according to claim 1 which contains 4 —N═O groups.

6. A piperazinyl pyrimidinyl nitroso compound according to claim 1 where the pharmaceutically acceptable salt is selected from the group consisting of salts of the following acids acetic, aspartic, benzenesulfonic, benzoic, bicarbonic, bisulfuiric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic.

7. A piperazinyl pyrimidinyl nitroso compound according to claim 1 where (P)—R_{N} is —N═O.

8. A piperazinyl pyrimidinyl nitroso compound according to claim 7 where the substituted pyrimidinyl nitroso compound is
5-nitroso-2,4-di(1-pyrrolidinyl)-6-(4-nitroso-1-piperazinyl)pyrimidine.

9. A piperazinyl pyrimidinyl nitroso compound according to claim 1 where (P)—R_{N} is (P)—R_{N-1}—O—OC—(CH₂)ₙ₁.

10. A piperazinyl pyrimidinyl nitroso compound according to claim 9 where the piperazinyl pyrimidinyl nitroso compound is
5-nitroso-2,4-di(1-pyrrolidinyl)-6-[4-(3-propionic acid methyl ester)piperazin-1-yl]pyrimidine.

11. N-nitrosometoprolol compound of formula (IV) 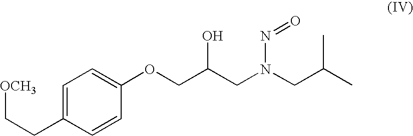
and pharmaceutically acceptable salts thereof

12. N-nitrosometoprolol compound (IV) according to claim 11 where the pharmaceutically acceptable salt is selected from the group consisting of salts of the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic.

13. A method of treating a human who has an ischemic disease selected from the group consisting of coronary heart disease, stroke, hemorrhagic shock, peripheral vascular disease (upper and lower extremities) and transplant related injuries and who is in need of treatment which comprises administering to that human an anti-ischemic effective amount of a piperazinyl pyrimidinyl nitroso compound of formula (II) 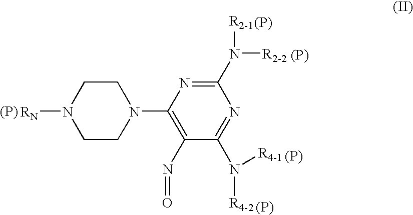
where (P)—R_{N} is:
—N═O,
(P)—R_{N-1}—O—OC—(CH₂)ₙ₁— where n, is 1 thru 6 and where (P)—R_{N-1} is H— or C₁-C₄ alkyl,
C₁-C₆ alkyl,
where (P)—R₂₋₁ is:
—N═O and
C₁-C₆ alkyl;
where (P)—R₂₋₂ is:
C₁-C₆ alkyl; and
where (P)—R₂₋₁ and (P)—R₂₋₂ are taken together with the attached nitrogen atom to form a ring selected from the group consisting of:
pyrrolidinyl,
piperidinyl,
homopiperidinyl,
morpholinyl,
4-nitroso-1-piperazinyl;
where (P)—R₄₋₁ is
—N═O and
C₁-C₆ alkyl; and
where (P)—R₄₋₂ is
C₁-C₆ alkyl; and
where (P)—R₄₋₁ and (P)—R₄₋₂ are taken together with the attached nitrogen atom to form a ring selected from the group consisting of:
pyrrolidinyl,
piperidinyl,
homopiperidinyl,
morpholinyl,
4-nitroso-1-piperazinyl;
or N-nitrosometoprolol of formula (IV) 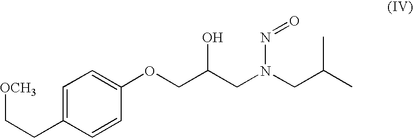
and pharmaceutically acceptable salts thereof.

14. A method of treating a human who has an ischemic disease according to claim 13 where the administering is IV or oral.

15. A method of treating a human who has an ischemic disease according to claim 13 where the IV anti-ischemic effective amount is from about 5 to about 100 mg/kg/dose.

16. A method of treating a human who has an ischemic disease according to claim 13 where the oral anti-ischemic effective amount is from about 5 to about 50 mg/kg/dose.

17. A method of treating a human who has an ischemic disease according to claim 13 where the compound is a piperazinyl pyrimidinyl nitroso compound of formula (II).

18. A method of treating a human who has an ischemic disease according to claim 17 where the piperazinyl pyrimidinyl nitroso compound (II) is
5-nitroso-2,4-di(1-pyrrolidinyl)-6-(4-nitroso-1-piperazinyl)pyrimidine or
5-nitroso-2,4-di(1-pyrrolidinyl)-6-[4-(3-propionic acid methyl ester)piperazin-1-yl]pyrimidine
